(19) United States
(12) Patent Application Publication
(10) Pub. No.: US 2023/0405091 A1
(43) Pub. Date: Dec. 21, 2023

(54) METHODS OF MODULATING GASTROINTESTINAL METABOLITES

(71) Applicants: DSM IP ASSETS B.V., Heerlen (NL); NOVOZYMES A/S, Bagsvaerd (DK)

(72) Inventors: Joshua CLAYPOOL, Kaiseraugst (CH); Ghislain SCHYNS, Kaiseraugst (CH)

(21) Appl. No.: 18/248,616

(22) PCT Filed: Oct. 15, 2021
(86) PCT No.: PCT/EP2021/078609

§ 371 Date: Apr. 11, 2023

Related U.S. Application Data
(60) Provisional application No. 63/092,082, filed on Oct. 15, 2020.

(51) Int. Cl. A61K 38/44; A23K 20/189; A23K 20/147; A23K 50/30; A23K 50/75

(52) U.S. Cl. CPC A61K 38/446; C12Y 115/01001; A23K 20/189; A23K 20/147; A23K 50/30; A23K 50/75

(57) ABSTRACT

The present disclosure relates to methods of feeding animals by providing feed additives that modulate the gut microbiome to improve the health, nutrition, and growth performance. The present disclosure further relates to methods of modulating metabolites present in the gastrointestinal tract of an animal. Such modulation includes, for example, modulating the level said metabolites.

This application contains a Sequence Listing in computer readable form, which is incorporated herein by reference.

### BACKGROUND

The gut microbiome, e.g., bacteria, viruses, fungi, mold, protozoa, etc. that reside in the digestive track, is responsible for converting undigested and unabsorbed components of an animal's diet into thousands of biologically active metabolites. These metabolites interface in turn with the local and systemic physiology of the animal as well as the animal's external environment.

Under normal circumstances, the biochemical output of the microbiome is dictated in part by the composition of food consumed by the animal and in part by the phylogenetic composition of the gut microbiome. In a conventional diet, particularly one comprising plant-fiber polysaccharides and species such as cellulose, lignin, hemicellulose, pectins, and starch-bound protein, a portion of the food consumed by the animal remains undigested and unabsorbed by the primary digestive process. These unabsorbed species reach the lower intestinal system, where they can be processed and utilized by the microbiota and converted to metabolites. The resulting gut metabolome generated by the metabolic action of the gut microbiome on these unabsorbed components of the feed is affected by the chemical compositions of those various unabsorbed components.

Metabolites produced in the gut can be absorbed, for example through the colonic or portal circulatory systems, and transported to other organs of the animal where they can affect the structure and/or function of those organs. These biochemicals in turn affect diverse biological functions, such as nutrient absorption, energy regulation, mitochondrial function, systemic inflammation, stress response, liver function, kidney function, cardiometabolic function, satiety, mood, and alertness. Metabolites produced in the gut can also be excreted by the animal to its external environment.

In some cases, the metabolites produced by the gut microbiome are beneficial to the host or otherwise contribute to the productivity, health, welfare and sustainability of the host animal. In other cases, the metabolites produced by the gut microbiome are detrimental to the host and result in decreased productivity, health, or welfare. Certain metabolites are undesirable because they are detrimental to the external environment of the animal when excreted, and can result in water, soil, and/or atmospheric pollution, or otherwise increase the environmental footprint of raising the animals.

Overall animal productivity and health are critical factors in the economics of the animal protein production industry. Consumer and regulatory pressure for improved sustainability are increasingly essential to maintaining competitiveness in the production industry.

There is thus a need to be able to modulate or otherwise control the metabolic pathways and metabolic output of the gut microbiome in animals for the purpose of improving nutrition, health, welfare and/or sustainability of production animals and companion animals. The challenge, however, is that animals typically exhibit high taxonomic variability in the phylogenetic composition of their gut microbiomes. Consequently, conventional feed additives that target the gut microbiota are generally understood in the industry to provide inconsistent effects on the gut metabolomes of the animals to which they are fed.

Surprisingly, the inventors of the present invention found that superoxide dismutases (SOD) provide beneficial effects when used in an animal feed or as an animal feed additive. In particular Feed supplied SODs can modify/modulate the abundance of enzymes in the microbiome metabolic pathways associated with central carbon and central nitrogen utilization. For example supplied SOD preparations according to the invention can modify/modulate the abundance of enzymes in the microbiome responsible for pathway to produce propanoate from pyruvic acid. The supplied enzymes can therefore improve performance of animal production via modulation of pathways present in the microbiome.

Superoxide dismutase (SOD, EC 1.15.1.1) is an enzyme that alternately catalyzes the dismutation (or partitioning) of the superoxide (02) radical into either ordinary molecular oxygen (O₂) or hydrogen peroxide (H₂O₂). Superoxide is produced as a by-product of oxygen metabolism and, if not regulated, causes many types of cell damage. Thus, SOD is an important antioxidant defense in nearly all living cells exposed to oxygen. SODs are used in the pharmaceutical, cosmetic, food, and environmental protection industries due to their excellent antioxidant properties. Historically, SODs were isolated from animal or plant sources, but the microbial sources organisms can be easily induced and cultivated on a large scale

SODs naturally occur in many organisms such as plants, insects, birds, reptiles and mammals. Four types of SODs have been reported according to their metal cofactors: manganese SOD (Mn-SOD), iron SOD (Fe-SOD), copper/zinc SOD (Cu/Zn-SOD), and nickel SOD (Ni-SOD)₂.

For example, mammalian (bovine) SOD and bacterial (E. coli) Mn-SOD (S5639) are commercially available from Sigma. Preferred superoxide dismutases are SODs of fungal origin.

### SUMMARY

In one aspect, provided herein are methods of improving the nutrition, health, welfare, and sustainability of animals by providing to the animals feed additives that increase or decrease the expression of one or more metabolic pathways in the metagenome of the animal's microbiome.

In certain embodiments, the methods of improving the nutrition of the animal comprise increasing the abundance of, expression of, or flux through metabolic pathways in the metagenome of the gastrointestinal microflora that are responsible for harvesting nutritional energy from undigested components of the animal's diet.

In one embodiment, the invention is related to a method of improving nitrogen utilization in an animal, the method comprising:
a. administering a nutritional composition comprising a base nutritional composition and a SOD preparation to said animal,
b. wherein said SOD preparation comprises a polypeptide having superoxide dismutase (SOD) activity; and
c. wherein the level of a plurality of metabolites associated with enhanced nitrogen utilization is higher in a gastrointestinal sample from said animal compared to a gastrointestinal sample from a comparable control animal that has been administered a comparable nutritional composition comprising said base nutritional composition and lacking said SOD preparation.

In a second embodiment, the invention is related to a method of improving carbon utilization in an animal, the method comprising:
d. administering a nutritional composition comprising a base nutritional composition and a SOD preparation to said animal,
e. wherein said SOD preparation comprises a polypeptide having superoxide dismutase (SOD) activity; and
f. wherein the level of a plurality of metabolites associated with improved carbon utilization in a gastrointestinal sample from said animal is higher compared to a comparable control animal that has been administered a comparable nutritional composition comprising said base nutritional composition and lacking said SOD preparation.

In a third embodiment of the invention, said plurality comprises at least 2, 3, 4, 5, 6, 7, 8, 9, or 10 metabolites selected from the group consisting of (R)-lactate, (R)-lactoyl-CoA, (S)-lactate, (S)-propane-1,2,-diol, 1-propanal, acetate, acetyl-CoA, acryloyl-CoA, propanoate, propanoyl-CoA, and pyruvate.

The invention is related to the use of one or more polypeptides having SOD activity in animal feed as defined above, wherein in a preferred embodiment, the SOD is an EC 1.15.1.1 SOD, for example a superoxide dismutase of microbial origin.

The SOD according to the invention is preferably selected from the group consisting of a Cu-SOD, a Zn-SOD, a Mn-SOD, and an Fe-SOD.

A commercially relevant aspect of the invention is directed to an animal feed additive comprising one or more polypeptides having superoxide dismutase (SOD) activity wherein the polypeptide having superoxide dismutase activity is selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1.
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 52 to 170 of SEQ ID NO: 1.
f. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 55 to 136 of SEQ ID NO: 2;
g. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 12 to 149 of SEQ ID NO: 3;
h. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 47 to 179 of SEQ ID NO: 4;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the sequence of SEQ ID NO: 5;
j. a polypeptide encoded by a polynucleotide that hybridizes under medium-high stringency conditions with coding sequence of SEQ ID NO: 5 or the full-length complement thereof;
k. a variant of the polypeptide of SEQ ID NO: 1, or SEQ ID NO: 2, or SEQ ID NO: 3, or SEQ ID NO: 4, or amino acid residues 52 to 170 of SEQ ID NO: 1, or amino acid residues 55 to 136 of SEQ ID NO: 2, or amino acid residues 12 to 149 of SEQ ID NO: 3, or amino acid residues 47 to 179 of SEQ ID NO: 4 comprising a substitution, deletion, and/or insertion at one or more (e.g., several) positions and that has superoxide dismutase activity; and
l. a fragment of the polypeptide of (a), (b), (c), (d), (e), (f), (g), (h), (i), (j), or (k) that has superoxide dismutase activity.

### OVERVIEW OF SEQUENCE LISTING

SEQ ID NO 1 is a mature polypeptide having superoxide dismutase (SOD) activity from Trichoderman reesei comprising 307 amino acid residues wherein residues 1 to 20 make up the signal peptide and residues 52 to 170 make up the SOD.
SEQ ID NO 2 is a mature polypeptide having superoxide dismutase (SOD) activity from Aspergillus versicolor comprising 206 amino acid wherein residues 1 to 20 make up the signal peptide and residues 55 to 136 make up the SOD.
SEQ ID NO 3 is a mature polypeptide having superoxide dismutase (SOD) activity from Aspergillus deflectus comprising 154 amino acid residues wherein residues 1 to 16 make up the signal peptide and residues 12 to 149 make up the SOD
SEQ ID NO 4 is a mature polypeptide having superoxide dismutase (SOD) activity from Aspergillus egyptiacus comprising 188 amino acid residues wherein residues 1 to 19 make up the signal peptide and residues 47 to 179 make up the SOD.
SEQ ID NO 5 is a polynucleotide sequence coding sequence of the polypeptide of SEQ ID NO: 1.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

It is understood that terms such as “comprises,” “comprised,” “comprising,” and the like have the meaning attributed to it in U.S. patent law; i.e., they mean “includes,” “included,” “including,” and the like and are intended to be inclusive or open ended and does not exclude additional, unrecited elements or method steps; and that terms such as “consisting essentially of” and “consists essentially of” have the meaning ascribed to them in U.S. patent law; i.e., they allow for elements not explicitly recited, but exclude elements that are found in the prior art or that affect a basic or novel characteristic of the invention.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or B; A or C; B or C; A and B; A and C; B and C; A (alone); B (alone); and C (alone).

As used herein the term “administering” includes providing a SOD preparation, a nutritional composition, a liquid, or an animal feed composition described herein, to an animal such that the animal can ingest the SOD preparation, the nutritional composition, the liquid, or the animal feed composition. In such embodiments, the animal ingests some portion of the SOD preparation, the nutritional composition, or the animal feed composition. In some embodiments, the SOD preparation, the nutritional composition, the liquid, or the animal feed composition is provided to said animal such that the animal may ingest the SOD preparation, the nutritional composition, the liquid, or the animal feed composition at will. In some embodiments, the SOD preparation, the nutritional composition, the liquid, or the animal feed composition is administered to said animal as a prescribed diet. In some embodiments, the SOD preparation, the nutritional composition, the liquid, or the animal feed composition is administered to said animal via manual feeding, e.g., an oral syringe feeding, a tube feeding, etc. In some embodiments, the SOD preparation, the nutritional composition, the liquid, or the animal feed composition is administered to said animal oral, e.g., at will or manually. In some embodiments, the animal ingests some portion of the SOD preparation, the nutritional composition, the liquid, or the animal feed composition in every 24-hour period or every other 24-hour period for at least 7 days, 14 days, 21 days, 30 days, 45 days, 60 days, 75 days, 90 days or 120 days. In some embodiments, the SOD preparation may be dissolved in water or another liquid, and the animal ingests some portion of the SOD preparation by drinking the liquid. In some embodiments, the oligosaccharide is provided to the animal via its drinking water. In some embodiments, the SOD preparation, nutritional composition, liquid, or animal feed composition is consumed at will.

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pets such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Feed Conversion Ratio (FCR): FCR is a measure of an animal's efficiency in converting feed mass into increases of the desired output. Animals raised for meat—such as swine, poultry and fish—the output is the mass gained by the animal. Specifically FCR is calculated as feed intake divided by weight gain, all over a specified period. Improvement in FCR means reduction of the FCR value. A FCR improvement of 2% means that the FCR was reduced by 2%.

Feed Premix: The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

European Production Efficiency Factor (EPEF): The European Production Efficiency Factor is a way of comparing the performance of animals. This single-figure facilitates comparison of performance within and among farms and can be used to assess environmental, climatic and animal management variables. The EPEF is calculated as [(liveability (%)×Liveweight (kg))/(Age at depletion (days)×FCR)]×100, wherein livability is the percentage of animals alive at slaughter, Liveweight is the average weight of the animals at slaughter, age of depletion is the age of the animals at slaughter and FCR is the feed conversion ratio at slaughter.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide or a catalytic domain having one or more (e.g., several) amino acids absent from the amino and/or carboxyl terminus of a mature polypeptide or domain; wherein the fragment has SOD activity.several) amino acids absent from the amino and/or carboxyl terminus of a mature polypeptide or domain; wherein the fragment has SOD activity.

In one aspect, a fragment of a GH25 SOD (such as one of SEQ ID NO: 1 to 5) comprises at least 180 amino acids, such as at least 185 amino acids, at least 190 amino acids, at least 195 amino acids, at least 200 amino acids, at least 205 amino acids or at least 210 amino acids and has SOD activity. In another aspect, a fragment of a GH25 SOD (such as one of SEQ ID NO: 1 to 5) comprises at least 90% of the length of the mature polypeptide, such as at least 92%, at least 94%, at least 96%, at least 98% or at least 99% of the length of the mature polypeptide and has SOD activity.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Obtained or obtainable from: The term “obtained or obtainable from” means that the polypeptide may be found in an organism from a specific taxonomic rank. In one embodiment, the polypeptide is obtained or obtainable from the kingdom Fungi, wherein the term kingdom is the taxonomic rank. In a preferred embodiment, the polypeptide is obtained or obtainable from the phylum Ascomycota, wherein the term phylum is the taxonomic rank. In another preferred embodiment, the polypeptide is obtained or obtainable from the subphylum Pezizomycotina, wherein the term subphylum is the taxonomic rank. In another preferred embodiment, the polypeptide is obtained or obtainable from the class Eurotiomycetes, wherein the term class is the taxonomic rank.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. The skilled person can also compare the sequence to those of the application as filed. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Roughage: The term “roughage” means dry plant material with high levels of fiber, such as fiber, bran, husks from seeds and grains and crop residues (such as stover, copra, straw, chaff, sugar beet waste).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Variant: The term “variant” means a polypeptide having SOD activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a SOD variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the SOD activity of the parent SOD, such as SEQ ID NO: 1 to 5.

Nutrient: The term “nutrient” in the present invention means components or elements contained in dietary feed for an animal, including water-soluble ingredients, fat-soluble ingredients and others. The example of water-soluble ingredients includes but is not limited to carbohydrates such as saccharides including glucose, fructose, galactose and starch; minerals such as calcium, magnesium, zinc, phosphorus, potassium, sodium and sulfur; nitrogen source such as amino acids and proteins, vitamins such as vitamin B1, vitamin B2, vitamin B3, vitamin B6, folic acid, vitamin B12, biotin and phatothenic acid. The example of the fat-soluble ingredients includes but is not limited to fats such as fat acids including saturated fatty acids (SFA); mono-unsaturated fatty acids (MUFA) and poly-unsaturated fatty acids (PUFA), fibre, vitamins such as vitamin A, vitamin E and vitamin K.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

I. SOD Preparations Used as Animal Microbiome Metabolic Modulators

Disclosed herein are microbiome metabolic modulators comprising SOD preparations that exhibit complex functional modulation of a microbial community, such as the animal gut microbiome. The SOD preparations provide a utility to modulate, modify, or regulate the utilization of fermentable carbon by microflora and direct metabolic flux to beneficial species, thus providing a microbiome-mediated health or nutritional benefit.

SOD compositions capable of complex modulation of the microbiota of animals have utility as feed additives that improve animal health and nutrition via their impact on the animal microbiome. For example, modulation of butyrate production by the gut microflora confers health benefits to the animal by promoting a healthy gut mucosa, barrier function, and via anti-inflammatory effects. Modulation of propionic acid production affects the metabolic energy extracted from the animal's diet via increased host gluconeogenesis. Relevant microbial communities include, for example, ileal, jejunal, and cecal and/or fecal microbiota in poultry, pigs, dogs, cats, horses, or the ruminant microbiota of cattle, cows, sheep, etc. Other microbial communities include the skin microflora, nasal microflora, etc.

The term “gut” as used herein designates the gastrointestinal or digestive tract (also referred to as the alimentary canal) and it refers to the system of organs within multi-cellular animals which takes in food, digests it to extract energy and nutrients, and expels the remaining waste.

The term gut “microflora” as used herein refers to the natural microbial cultures residing in the gut and maintaining health by aiding in proper digestion.

The term “modulate” as used herein in connection with the gut microflora generally means to change, manipulate, alter, or adjust the function or status thereof in a healthy and normally functioning animal, i.e. a non-therapeutic use.

II. SOD Preparation

The polypeptide having SOD activity is preferably dosed at a level of 100 to 2000 mg enzyme protein per kg animal feed, such as 200 to 1800 mg, 300 to 1500 mg, 400 to 1200 mg, 500 to 900 mg, 600 to 800 mg enzyme protein per kg animal feed, or any combination of these intervals.

The animal feed to which the SOD according to the invention is added comprises a protein source and an energy source.

The protein source of the animal feed is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

The energy source of the animal feed is selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

In a preferred example, the animal feed further comprises one or more components selected from the list consisting of one or more additional enzymes; one or more microbes; one or more vitamins; one or more minerals; one or more amino acids; and one or more other feed ingredients, as described herein.

The amino acid changes in the SOD sequences disclosed above may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in the polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for SOD activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Enzyme Formulation

The polypeptide having SOD activity of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus, in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

In one embodiment, the liquid formulation further comprises 20%-80% polyol (i.e. total amount of polyol), preferably 25%-75% polyol, more preferably 30%-70% polyol, more preferably 35%-65% polyol or most preferably 40%-60% polyol. In one embodiment, the liquid formulation comprises 20%-80% polyol, preferably 25%-75% polyol, more preferably 30%-70% polyol, more preferably 35%-65% polyol or most preferably 40%-60% polyol wherein the polyol is selected from the group consisting of glycerol, sorbitol, propylene glycol (MPG), ethylene glycol, diethylene glycol, triethylene glycol, 1, 2-propylene glycol or 1, 3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e. total amount of polyol), preferably 25%-75% polyol, more preferably 30%-70% polyol, more preferably 35%-65% polyol or most preferably 40%-60% polyol wherein the polyol is selected from the group consisting of glycerol, sorbitol and propylene glycol (MPG).

In one embodiment, the liquid formulation further comprises preservative, preferably selected from the group consisting of sodium sorbate, potassium sorbate, sodium benzoate and potassium benzoate or any combination thereof. In one embodiment, the liquid formulation comprises 0.02% to 1.5% w/w preservative, more preferably 0.05% to 1.0% w/w preservative or most preferably 0.1% to 0.5% w/w preservative. In one embodiment, the liquid formulation comprises 0.001% to 2.0% w/w preservative (i.e. total amount of preservative), preferably 0.02% to 1.5% w/w preservative, more preferably 0.05% to 1.0% w/w preservative or most preferably 0.1% to 0.5% w/w preservative wherein the preservative is selected from the group consisting of sodium sorbate, potassium sorbate, sodium benzoate and potassium benzoate or any combination thereof.

For a solid formulation, the formulation may be for example as a granule, spray dried powder or agglomerate (e.g. as disclosed in WO2000/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In one embodiment, the composition is a solid composition, such as a spray dried composition, comprising the polypeptide having SOD activity of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate, magnesium sulfate and calcium carbonate.

The present invention also relates to enzyme granules/particles comprising the polypeptide having SOD activity of the invention optionally combined with one or more additional enzymes. The granule is composed of a core, and optionally one or more coatings (outer layers) surrounding the core.

Typically, the granule/particle size, measured as equivalent spherical diameter (volume based average particle size), of the granule is 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier. Preparation methods include known feed and granule formulation technologies, e.g.:
a) spray dried products, wherein a liquid enzyme-containing solution is atomized in a spray drying tower to form small droplets which during their way down the drying tower dry to form an enzyme-containing particulate material;
b) layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606;
c) absorbed core particles, wherein rather than coating the enzyme as a layer around the core, the enzyme is absorbed onto and/or into the surface of the core. Such a process is described in WO 97/39116.
d) extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme;
e) prilled products, wherein an enzyme-containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique;
f) mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibres to reinforce the particles to give the so-called T-granulate. Reinforced particles, being more robust, release less enzymatic dust.
g) size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons);
h) fluid bed granulation, which involves suspending particulates in an air stream and spraying a liquid onto the fluidized particles via nozzles. Particles hit by spray droplets get wetted and become tacky. The tacky particles collide with other particles and adhere to them and form a granule;
i) the cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% w/w water.

The core may include additional materials such as fillers, fibre materials (cellulose or synthetic fibres), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

In one embodiment, the core comprises a material selected from the group consisting of salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In one embodiment, the core comprises a clay mineral such as kaolinite or kaolin.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt and/or wax and/or flour coating, or other suitable coating materials.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In some embodiments the thickness of the coating is below 100 am, such as below 60 μm, or below 40 μm.

The coating should encapsulate the core unit by forming a substantially continuous layer. A substantially continuous layer is to be understood as a coating having few or no holes, so that the core unit is encapsulated or enclosed with few or no uncoated areas. The layer or coating should in particular be homogeneous in thickness.

The coating can further contain other materials as known in the art, e.g., fillers, antisticking agents, pigments, dyes, plasticizers and/or binders, such as titanium dioxide, kaolin, calcium carbonate or talc.

The granule may comprise a core comprising the polypeptide having SOD activity of the invention, one or more salt coatings and one or more wax coatings. Examples of enzyme granules with multiple coatings are shown in WO1993/07263, WO1997/23606 and WO2016/149636.

A salt coating may comprise at least 60% by weight of a salt, e.g., at least 65%, at least 70%, at least 75%, at least 80%, at least 85%, at least 90%, at least 95% or at least 99% by weight.

The salt may be added from a salt solution where the salt is completely dissolved or from a salt suspension wherein the fine particles are less than 50 am, such as less than 10 am or less than 5 μm.

The salt coating may comprise a single salt or a mixture of two or more salts. The salt may be water soluble, in particular having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, sorbate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO1997/05245, WO1998/54980, WO1998/55599, WO2000/70034, WO2006/034710, WO2008/017661, WO2008/017659, WO2000/020569, WO2001/004279, WO1997/05245, WO2000/01793, WO2003/059086, WO2003/059087, WO2007/031483, WO2007/031485, WO2007/044968, WO2013/192043, WO2014/014647 and WO2015/197719 or polymer coating such as described in WO 2001/00042.

Specific examples of suitable salts are NaCl (CH20° C.=76%), Na2CO3 (CH20° C.=92%), NaNO3 (CH20° C.=73%), Na2HPO4 (CH20° C.=95%), Na3PO4 (CH25° C.=92%), NH4Cl (CH20° C.=79.5%), (NH4)2HPO4 (CH20° C.=93,0%), NH4H2PO4 (CH20° C.=93.1%), (NH4)2SO4 (CH20° C.=81.1%), KCl (CH20° C.=85%), K2HPO4 (CH20° C.=92%), KH2PO4 (CH20° C.=96.5%), KNO3 (CH20° C.=93.5%), Na2SO4 (CH20° C.=93%), K2SO4 (CH20° C.=98%), KHSO4 (CH20° C.=86%), MgSO4 (CH20° C.=90%), ZnSO4 (CH20° C.=90%) and sodium citrate (CH25° C.=86%). Other examples include NaH2PO4, (NH4)H2PO4, CuSO4, Mg(NO3)2, magnesium acetate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, sodium acetate, sodium benzoate, sodium citrate, sodium sulfate, zinc acetate, zinc benzoate, zinc carbonate, zinc chloride, zinc citrate and zinc sorbate.

The salt may be in anhydrous form, or it may be a hydrated salt, i.e. a crystalline salt hydrate with bound water(s) of crystallization, such as described in WO 99/32595. Specific examples include anhydrous sodium sulfate (Na2SO4), anhydrous magnesium sulfate (MgSO4), magnesium sulfate heptahydrate (MgSO4·7H2O), zinc sulfate heptahydrate (ZnSO4·7H2O), sodium phosphate dibasic heptahydrate (Na2HPO4·7H2O), magnesium nitrate hexahydrate (Mg(NO3)2(6H2O)), sodium citrate dihydrate and magnesium acetate tetrahydrate.

Preferably the salt is applied as a solution of the salt, e.g., using a fluid bed.

A wax coating may comprise at least 60% by weight of a wax, e.g., at least 65%, at least 70%, at least 75%, at least 80%, at least 85%, at least 90%, at least 95% or at least 99% by weight.

Specific examples of waxes are polyethylene glycols; polypropylenes; Carnauba wax; Candelilla wax; bees wax; hydrogenated plant oil or animal tallow such as polyethylene glycol (PEG), methyl hydroxy-propyl cellulose (MHPC), polyvinyl alcohol (PVA), hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil.

Non-dusting granulates may be produced, e.g., as disclosed in U.S. Pat. Nos. 4,106,991 and 4,661,452 and may optionally be coated by methods known in the art. The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Animal Feed

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore, such fish diets usually have a crude fat content of 200-310 g/kg.

An animal feed composition according to the invention has a crude protein content of 50-800 g/kg, and furthermore comprises one or more polypeptides having SOD activity as described herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein as defined above.

The animal feed composition of the invention may also contain animal protein, such as Meat and Bone Meal, Feather meal, and/or Fish Meal, typically in an amount of 0-25%. The animal feed composition of the invention may also comprise Dried Distillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) SOD/enzyme preparation may also be added before or during the feed ingredient step. Typically a liquid enzyme preparation comprises the SOD of the invention optionally with a polyol, such as glycerol, ethylene glycol or propylene glycol, and is added after the pelleting step, such as by spraying the liquid formulation onto the pellets. The SOD may also be incorporated in a feed additive or premix.

In an embodiment, the composition comprises one or more additional enzymes. In an embodiment, the composition comprises one or more microbes. In an embodiment, the composition comprises one or more vitamins. In an embodiment, the composition comprises one or more minerals. In an embodiment, the composition comprises one or more amino acids. In an embodiment, the composition comprises one or more other feed ingredients.

In another embodiment, the composition comprises one or more of the polypeptides of the invention, one or more formulating agents and one or more additional enzymes. In an embodiment, the composition comprises one or more of the polypeptides of the invention, one or more formulating agents and one or more microbes. In an embodiment, the composition comprises one or more of the polypeptides of the invention, one or more formulating agents and one or more vitamins. In an embodiment, the composition comprises one or more of the polypeptides of the invention and one or more minerals. In an embodiment, the composition comprises the polypeptide of the invention, one or more formulating agents and one or more amino acids. In an embodiment, the composition comprises one or more of the polypeptides of the invention, one or more formulating agents and one or more other feed ingredients.

In a further embodiment, the composition comprises one or more of the polypeptides of the invention, one or more formulating agents and one or more components selected from the list consisting of: one or more additional enzymes; one or more microbes; one or more vitamins; one or more minerals; one or more amino acids; and one or more other feed ingredients.

The final SOD concentration in the diet is within the range of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

The final SOD concentration in the diet can also be determined in Units/kg feed, which is within the range of 100 to 3000 Units per kg animal feed, such as 200 to 3000 U/kg, 300 to 2000 U/kg, 100 to 800 U/kg, 100 to 400 U/kg, or any combination of these intervals.

In another embodiment, the compositions described herein optionally include one or more enzymes for improving feed digestibility. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Thus the composition of the invention may also comprise at least one other enzyme selected from the group comprising of acetylxylan esterase (EC 3.1.1.23), acylglycerol lipase (EC 3.1.1.72), alpha-amylase (EC 3.2.1.1), beta-amylase (EC 3.2.1.2), arabinofuranosidase (EC 3.2.1.55), cellobiohydrolases (EC 3.2.1.91), cellulase (EC 3.2.1.4), feruloyl esterase (EC 3.1.1.73), galactanase (EC 3.2.1.89), alpha-galactosidase (EC 3.2.1.22), beta-galactosidase (EC 3.2.1.23), beta-glucanase (EC 3.2.1.6), beta-glucosidase (EC 3.2.1.21), triacylglycerol lipase (EC 3.1.1.3), lysophospholipase (EC 3.1.1.5), alpha-mannosidase (EC 3.2.1.24), beta-mannosidase (mannanase) (EC 3.2.1.25), phytase (EC 3.1.3.8, EC 3.1.3.26, EC 3.1.3.72), phospholipase A1 (EC 3.1.1.32), phospholipase A2 (EC 3.1.1.4), phospholipase D (EC 3.1.4.4), pullulanase (EC 3.2.1.41), pectinesterase (EC 3.1.1.11), beta-xylosidase (EC 3.2.1.37), or any combination thereof.

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

In one embodiment, the amount of vitamins is 0.001% to 10% by weight of the composition. In one embodiment, the amount of minerals is 0.001% to 10% by weight of the composition.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

III. Selectively Promoting or Inhibiting Production of Gastrointestinal Metabolites

A. Gastrointestinal Metabolites

In certain embodiments, the methods described herein include selectively promoting or inhibiting the production of one or more gastrointestinal metabolites in an animal. In some embodiments, one or more of the metabolites are detected and quantified. Metabolites include but are not limited to metabolites associated with the C3 microbiome pathway e.g., (R)-lactate, (R)-lactoyl-CoA, (S)-lactate, (S)-propane-1,2,-diol, 1-propanal, acetate, acetyl-CoA, acryloyl-CoA, propanoate, propanoyl-CoA, and pyruvate; metabolites associated with the Energy Metabolism microbiome pathway e.g., 2-oxoglutarate, fumarate, L-alanine, L-glutamate, oxaloacetate, propanoyl-CoA, pyruvate, and succinate; metabolites associated with the Adverse Amino Acid Degradation microbiome pathway e.g., (3S,5S)-3,5-diaminohexanoate, (S)-3-methyl-2-oxopentanoate, (S)-5-amino-3-oxohexanoate, 2-oxoglutarate, acetyl-CoA, ammonia, D-alanine, Formate, Fumarate, Glycine, L-2-amino-3-oxobutanoate, L-alanine, L-asparagine, L-aspartate, L-glutamate, L-isoleucine, N-formimino-L-glutamate, N-formyl-L-glutamate, N2-succinylglutamate, and Pyruvate; and metabolites associated with the C4 Pathway microbiome pathway e.g., (3R)-3-hydroxybutanoyl-CoA, (R)-lactate, (R)-lactoyl-CoA, (S)-3-aminobutanoyl-CoA, (S)-3-hydroxy-isobutanoate, (S)-3-hydroxy-isobutanoyl-CoA, (S)-3-hydroxybutanoyl-CoA, (S)-5-amino-3-oxohexanoate, (S)-lactate, 4-hydroxybutanoate, Acetate, Acetoacetate, acetoacetyl-CoA, acetyl-CoA, butanoate, butanoyl-CoA, coenzyme_A, crotonyl-CoA, succinate, succincate_semialdehyde, and succinyl-CoA.

In some embodiments, one or more of the metabolites promote growth of the animal. In certain embodiments, the methods described herein include promoting or inhibiting the production of one or more gastrointestinal metabolites in an animal.

B. Sampling and Detecting Gastrointestinal Metabolites

Gastrointestinal samples can be obtained from an animal in any standard form which reflects the metabolic contents of the gastrointestinal tract of the animal. Gastrointestinal samples include gastrointestinal tissue samples obtained e.g., by endoscopic biopsy. Gastrointestinal tissues include, e.g., oral tissue, esophagus, stomach, intestine, ileum, cecum, colon or rectum. Samples also feces, saliva, and gastrointestinal ascites. Methods of obtaining gastrointestinal samples are standard and known to the skilled artisan.

In some embodiments, the sample is a single sample from a single animal. In some embodiments, the sample is a combination of multiple samples from a single animal. In some embodiments, metabolites are purified from the sample prior to analysis. In some embodiments, metabolites from a single sample are purified. In some embodiments, metabolites from multiple samples from a single animal are purified and subsequently combined prior to analysis.

The metabolites that are present in gastrointestinal samples collected from animals or in fresh or spent culture media may be determined using methods described herein and known to the skilled artisan. Such methods include for example chromatography (e.g., gas (GC) or liquid chromatography (LC)) combined with mass spectrometry or NMR (e.g., 1H-NMR). The measurements may be validated by running metabolite standards through the same analytical systems.

In the case of gas chromatography-mass spectrometry (GC-MS) or liquid-chromatography-mass spectrometry (LC-MS) analysis, polar metabolites and fatty acids could be extracted using monophasic or biphasic systems of organic solvents and an aqueous sample and derivatized. An exemplary protocol for derivatization of polar metabolites involves formation of methoxime-tBDMS derivatives through incubation of the metabolites with 2% methoxylamine hydrochloride in pyridine followed by addition of N-tert-butyldimethylsilyl-N-methyltrifluoroacetamide (MTBSTFA) with 1% tert-butyldimethylchlorosilane (t-BDMCS). Non-polar fractions, including triacylglycerides and phospholipids, may be saponified to free fatty acids and esterified to form fatty acid methyl esters, for example, either by incubation with 2% H₂SO₄ in methanol or by using Methyl-8 reagent (Thermo Scientific). Derivatized samples may then be analyzed by GC-MS using standard LC-MS methods, for example, a DB-35MS column (30 m×0.25 mm i.d.×0.25μη, Agilent J&W Scientific) installed on a gas chromatograph (GC) interfaced with a mass spectrometer (MS). Mass isotopomer distributions may be determined by integrating metabolite ion fragments and corrected for natural abundance using standard algorithms. In the case of liquid chromatography-mass spectrometry (LC-MS), polar metabolites may be analyzed using a standard benchtop LC-MS/MS equipped with a column, such as a SeQuant ZIC-Philic polymeric column (2.1×150 mm; EMD Millipore).

Exemplary mobile phases used for separation could include buffers and organic solvents adjusted to a specific pH value.

In combination or in the alternative, extracted samples may be analyzed by ¹H-nuclear magnetic resonance (¹H-NMR). Samples may be combined with isotopically enriched solvents such as D2O, optionally in the presence of a buffered solution (e.g., Na₂HPO₄, NaH₂PO₄ in D2O, pH 7.4). Samples may also be supplemented with a reference standard for calibration and chemical shift determination (e.g., 5 mM 2,2-dimethyl-2-silapentane-5-sulfonate sodium salt (DSS-d₆, Isotec, USA)). Prior to analysis, the solution may be filtered or centrifuged to remove any sediment or precipitates, and then transferred to a suitable NMR tube or vessel for analysis (e.g., a 5 mm NMR tube). ¹H-NMR spectra may be acquired on a standard NMR spectrometer, such as an Avance II+500 Bruker spectrometer (500 MHz) (Bruker, DE), equipped with a 5 mm QXI-Z C/N/P probe-head) and analyzed with spectra integration software (such as Chenomx NMR Suite 7.1; Chenomx Inc., Edmonton, AB). Alternatively, ¹H-NMR may be performed following other published protocols known in the art (see e.g., Chassaing et al., Lack of soluble fiber drives diet-induced adiposity in mice, Am J Physiol Gastrointest Liver Physiol, 2015; Bai et al., Comparison of Storage Conditions for Human Vaginal Microbiome Studies, PLoS ONE, 2012:e36934).

C. Beneficial Microbes

In some embodiments, the methods described herein include selectively enhancing or promoting the growth of one or more microbial (e.g., bacterial) species in the gastrointestinal tract of an animal. In some embodiments, the microbial (e.g., bacterial) species is beneficial to the animal (e.g., beneficial to the health). In some embodiments, the methods described herein include selectively enhancing or promoting the growth of one or more microbial (e.g., bacterial) species in the gastrointestinal tract of an animal, wherein the microbial species produces one or more selected metabolites. In some embodiments, the microbial species is an archaea species.

In other embodiments, the microbial species is a virus, bacteriophage, or protozoan species. In some embodiments, the microbial species is a bacterial species. In some embodiments, the microbial species is a fungi species.

Bacteria disclosed herein include, but are not limited to, organisms classified as genera Enterococcus, Lactobacillus, Propionibacterium, Bifidobacterium, and Streptococcus.

D. Sampling and Detecting Gastrointestinal Microbes

Gastrointestinal microbiota samples can be obtained from an animal in any standard form which reflects the microbial contents of the gastrointestinal tract of the animal. Gastrointestinal microbiota samples include gastrointestinal tissue samples obtained e.g., by endoscopic biopsy. Gastrointestinal tissues include, e.g., oral tissue, esophagus, stomach, intestine, ileum, cecum, colon or rectum. Samples also feces, saliva, and gastrointestinal ascites. Methods of obtaining gastrointestinal microbiota samples are standard and known to the skilled artisan.

In some embodiments, the sample is a single sample from a single animal. In some embodiments, the sample is a combination of multiple samples from a single animal. In some embodiments, microbes (e.g., bacteria, e.g., total bacteria) are purified from the sample prior to analysis. In some embodiments, microbes (e.g., bacteria) from a single sample are purified. In some embodiments, microbes (e.g., bacteria) from multiple samples from a single animal are purified and subsequently combined prior to analysis.

In some embodiments, total DNA or total RNA is isolated from the sample. Genomic DNA can be extracted from samples using standard techniques known to the skilled artisan, including commercially available kits, such as the Mo Bio Powersoil®-htp 96 Well Soil DNA Isolation Kit (Mo Bio Laboratories, Carlsbad, CA), the Mo Bio Powersoil® DNA Isolation Kit (Mo Bio Laboratories, Carlsbad, CA), or the QIAamp DNA Stool Mini Kit (QIAGEN, Valencia, CA) according to the manufacturer's instructions. RNA can be extracted from samples using standard assays known to the skilled artisan including commercially available kits, such as the RNeasy PowerMicrobiome Kit (QIAGEN, Valencia, CA) and RiboPure Bacterial RNA Purification Kit (Life Technologies, Carlsbad, CA). Another method for isolation of microbial (e.g., bacterial) RNA may involve enrichment of mRNA in purified samples of bacterial RNA through removal of tRNA. Alternatively, RNA may be converted to cDNA, which can be used to generate sequencing libraries using standard methods such as the Nextera XT Sample Preparation Kit (Illumina, San Diego, CA).

Identification and determination of the relative abundance of a microbial (e.g., bacterial) species in a sample may be determined by standard molecular biology methods known to the skilled artisan, including e.g., genetic analysis (e.g. DNA sequencing (e.g., full genome sequencing, whole genome shotgun sequencing (WSG)), RNA sequencing, PCR, quantitative PCR (qPCR)), serology and antigen analysis, microscopy, metabolite identification, gram staining, flow cytometry, immunological techniques, and culture based methods such as counting colony forming units.

In some embodiments, identification and relative abundance of a microbial (e.g., bacterial) species is determined by whole genome shot gun sequencing (WGS), wherein extracted DNA is fragmented into pieces of various lengths (from 300 to about 40,000 nucleotides) and directly sequenced without amplification. Sequence data can be generated using any sequencing technology including for example, but not limited to Sanger, Illumina, 454 Life Sciences, Ion Torrent, ABI, Pacific Biosciences, and/or Oxford Nanopore.

Sequencing libraries for microbial (e.g., bacterial) whole-genome sequencing (WGS) may be prepared from microbial (e.g., bacterial) genomic DNA. For genomic DNA that has been isolated from an animal sample, the DNA may optionally be enriched for microbial (e.g., bacterial) DNA using commercially available kits, for example, the NEBNext Microbiome DNA Enrichment Kit (New England Biolabs, Ipswich, MA) or other enrichment kit. Sequencing libraries may be prepared from the genomic DNA using commercially available kits as well, such as the Nextera Mate-Pair Sample Preparation Kit, TruSeq DNA PCR-Free or TruSeq Nano DNA, or the Nextera XT Sample Preparation Kit (Illumina, San Diego, CA) according to the manufacturer's instructions.

Alternatively, libraries can be prepared using other kits compatible with the Illumina sequencing platform, such as the NEBNext DNA Library Construction Kit (New England Biolabs, Ipswich, MA). Libraries may then be sequenced using standard sequencing technology including, but not limited to, a MiSeq, HiSeq or NextSeq sequencer (Illumina, San Diego, CA).

Alternatively, a whole genome shotgun fragment library prepared using standard methods in the art may be used. For example, the shotgun fragment library could be constructed using the GS FLX Titanium Rapid Library Preparation Kit (454 Life Sciences, Branford, CT), amplified using a GS FLX Titanium emPCR Kit (454 Life Sciences, Branford, CT), and sequenced following standard 454 pyrosequencing protocols on a 454 sequencer (454 Life Sciences, Branford, CT).

Nucleic acid sequences can be analyzed to define taxonomic assignments using sequence similarity and phylogenetic placement methods or a combination of the two strategies. A similar approach can be used to annotate protein names, protein function, transcription factor names, and any other classification schema for nucleic acid sequences. Sequence similarity based methods include BLAST, BLASTx, tBLASTn, tBLASTx, RDP-classifier, DNAclust, RapSearch2, DIAMOND, USEARCH, and various implementations of these algorithms such as QIIME or Mothur. These methods map a sequence read to a reference database and select the best match. Common databases include KEGG, MetaCyc, NCBI non-redundant database, Greengenes, RDP, and Silva for taxonomic assignments. For functional assignments, reads are mapped to various functional databases such as COG, KEGG, BioCyc, MetaCyc, and the Carbohydrate-Active Enzymes (CAZy) database. Microbial clades are assigned using software including MetaPhlAn.

In some embodiments, the bacterial constituents are identified by characterizing the DNA sequence of bacterial 16S small subunit ribosomal RNA gene (16S rRNA gene). 16S rRNA gene is approximately 1,500 nucleotides in length, and in general is highly conserved across organisms, but contain specific variable and hypervariable regions (V1-V9) that harbor sufficient nucleotide diversity to differentiate species- and strain-level taxa of most organisms. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature.

Composition of a bacterial community can be deduced by sequencing full 16S rRNA gene, or at least one of the VI, V2, V3, V4, V5, V6, V7, V8, and V9 regions of this gene or by sequencing of any combination of variable regions from this gene (e.g. VI-3 or V3-5). In one embodiment, the VI, V2, and V3 regions are used to characterize a microbiota. In another embodiment, the V3, V4, and V5 regions are used to characterize a microbiota. In another embodiment, the V4 region is used to characterize a microbiota.

Sequences that are at least 97% identical to each other are grouped into Operational Taxonomic Units (OTUs). OTUs that contain sequences with 97% similarity correspond to approximately species level taxa. At least one representative sequence from each OTU is chosen, and is used to obtain a taxonomic assignment for an OTU by comparison to a reference database of highly curated 16S rRNA gene sequences (such as Greengenes or SILVA databases). Relationship between OTUs in a microbial community could be deduces by constructing a phylogenetic tree from representative sequences from each OTU. Using known techniques, in order to determine the full 16S sequence or the sequence of any variable region of the 16S sequence, genomic DNA is extracted from a bacterial sample, the 16S rRNA (full region or specific variable regions) amplified using polymerase chain reaction (PCR), the PCR products are cleaned, and nucleotide sequences delineated to determine the genetic composition of 16S rRNA gene or a variable region of the gene. If full 16S sequencing is performed, the sequencing method used may be, but is not limited to, Sanger sequencing. If one or more variable regions is used, such as the V4 region, the sequencing can be, but is not limited to being performed using the Sanger method or using a next-generation sequencing method, such as an Illumina method. Primers designed to anneal to conserved regions of 16S rRNA genes (e.g., the 515F and 805R primers for amplification of the V4 region) could contain unique barcode sequences to allow characterizing multiple microbial communities simultaneously.

In addition to the 16S rRNA gene, a selected set of genes that are known to be marker genes for a given species or taxonomic group is analyzed to assess the composition of a microbial community. These genes are alternatively assayed using a PCR-based screening strategy. For example, various strains of pathogenic Escherichia coli are distinguished using genes that encode heat-labile (LTI, LTlla, and LTIIb) and heat-stable (STI and STII) toxins, verotoxin types 1, 2, and 2e (VT1, VT2, and VT2e, respectively), cytotoxic necrotizing factors (CNF1 and CNF2), attaching and effacing mechanisms (eaeA), enteroaggregative mechanisms (Eagg), and enteroinvasive mechanisms (Einv). The optimal genes to utilize to determine the taxonomic composition of a microbial community by use of marker genes are familiar to one with ordinary skill in the art of sequence based taxonomic identification.

In some embodiments, the identity of the microbial composition is characterized by identifying nucleotide markers or genes, in particular highly conserved genes (e.g., “house-keeping” genes), or a combination thereof. Using defined methods, DNA extracted from a bacterial sample will have specific genomic regions amplified using PCR and sequenced to determine the nucleotide sequence of the amplified products.

E. Functional Metagenomic Analysis

In certain embodiments, the methods described herein include detecting or quantifying one or more metagenomic functions (e.g., biochemical reactions, metabolic pathways, catabolic pathways) performed by the microbial (e.g., bacterial) species in the gastrointestinal microbiota of an animal. In certain embodiments, the expression of the metagenomic function is detected or quantified in a gastrointestinal microbiota sample from an animal. Gastrointestinal microbiota samples can be obtained from an animal in any standard form which reflects the microbial contents of the gastrointestinal tract of the animal. Gastrointestinal microbiota samples include gastrointestinal tissue samples obtained e.g., by endoscopic biopsy. Gastrointestinal tissues include, e.g., oral tissue, esophagus, stomach, intestine, ileum, cecum, colon or rectum. Samples also feces, saliva, and gastrointestinal ascites. Methods of obtaining gastrointestinal microbiota samples are standard and known to the skilled artisan.

The metabolic pathways can be analyzed by first analyzing the gastrointestinal microbiota samples whole genome sequencing (e.g., whole genome shotgun sequencing) and making taxonomic assignments against a database (e.g., MetaPhlAn2 (db_v20)) to produce a metagenome. The metagenomes obtained by the whole genome sequencing can be annotated by homology against a functionally-annotated catalog using methods known in the art.

In certain embodiments, the metagenomic function is a biochemical pathway coded for by genes within the genome of a single microorganism of the gastrointestinal microbiota. In other embodiments, the metagenomic function is a biochemical pathway coded for by the genes of multiple distinct microorganisms of the gastrointestinal microbiota. In certain embodiments, the metagenomic function comprises multiple biochemical reactions, each of which converts one or more reactant metabolites to one or more product metabolites. In certain embodiments, the biochemical reactions that convert a reactant metabolite to a product metabolite involves the production of an intermediate by one microorganism in the microbiota, followed by conversion of the intermediate to the product metabolite by a different microorganism in the microbiota.

In certain embodiments, the set of all possible biochemical reactions in the metagenome of the gastrointestinal microbiota is described as a metabolic network. In particular embodiments, the metabolic network is represented as a graph, wherein the nodes of the graph denote all the possible metabolites and metabolic intermediates and the edges of the graph denote all the possible biochemical reactions performed by the microbiota.

In some embodiments, the one or more biochemical reactions performed by the microbiota are catalyzed by enzymes expressed by the microbiota. In certain embodiments, the one or more enzymatic reactions may be identified by their Enzyme Commission (E.C.) number. In particular embodiments, the one or more enzyme-catalyzed biochemical reactions have E.C. numbers selected from the group consisting of 1.1.1.28, 1.3.1.95, 1.3.5.4, 1.3.8., 1.3.8.1, 1.3.8.5, 2.8.3., 2.8.3.1, 2.8.3.1, 2.8.3.12, 2.8.3.17, 2.8.3.18, 2.8.3.8, 2.8.3.9, 4.2.1., 4.2.1.112, 4.2.1.120, 4.2.1.150, 4.2.1.167, 4.2.1.17, 4.2.1.2, 4.2.1.22, 4.2.1.28, 4.2.1.34, 4.2.1.49, 4.2.1.54, 4.2.1.55.

In other embodiments, the one or more biochemical reactions performed by the microbiota may be designated by reference to standard databases of metabolic functions. In certain embodiments, the biochemical reactions are denoted by their corresponding KEGG database ID or BioCyc databased ID. In particular embodiments, the BioCyc IDs of the one or more biochemical reactions are selected from the set consisting of 1.1.1.178-RXN, 1.2.1.25-RXN, 1.2.1.27-RXN, 1.2.1.54-RXN, 1.2.3.13-RXN, 1.4.1.11-RXN, 1.4.1.11-RXN, 2-M ETHYLACYL-COA-DEHYDROGENASE-RXN, 2.1.3.1-RXN, 2.6.1.14-RXN, 2.6.1.57-RXN, 2.6.1.57-RXN, 2.6.1.57-RXN, 2.6.1.57-RXN, 2.8.3.17-RXN, 2.8.3.17-RXN, 2.8.3.17-RXN, 2.8.3.9-RXN, 2KETO-3METHYLVALERATE-RXN, 2KETO-3METHYLVALERATE-RXN, 3-HYDROXBUTYRYL-COA-DEHYDRATASE-RXN, PHENYLPYRUVATE-DECARBOXYLASE-RXN.

F. Beneficial Metagenomic Functions

In certain embodiments, the methods described herein pertain to increasing the expression of microbiome metagenomic functions that translate to a nutritional, health, or welfare benefit in the host animal. In some embodiments, the microbiome metagenomic functions comprise one or more metabolic pathways or groups of pathways (e.g., superpathways). In certain embodiments, the microbiome metagenomic function comprises pathways that produce metabolites that are beneficial to the host animal.

In certain embodiments, the beneficial microbiome metagenomic function comprises pathways and metabolites responsible for recovering metabolic energy from otherwise undigested or unutilized components of the animals' diets. In some variations, the undigested or unutilized components of the animals' diet comprises fiber, non-starch polysaccharides, digestion-resistant carbohydrates, hemicellulosic species, pectins, fiber-bound protein, fiber-bound micronutrients, and chelated minerals or metals. In certain embodiments, the beneficial microbiome metagenomic function is the “C3 Pathway” associated with the production of gluconeogenic metabolites, which can be absorbed by the animal and recovered as metabolic energy. In particular embodiments, the C3 Pathway is defined by the total abundance of genes in the metagenome annotated by the E.C. numbers selected from the list of E.C. numbers consisting of 1.1.1.27, 1.2.1.87, 1.3.1.95, 2.8.3.1, and 4.2.1.28. In particular embodiments, the C3 Pathway is defined by the total abundance of genes in the metagenome annotated by reactions selected from the list of reactions having the BioCyc reaction ID consisting of L-LACTATE-DEHYDROGENASE-RXN, PROPANEDIOL-DEHYDRATASE-RXN, RXN-12736, RXN-8568, RXN-8807. In particular embodiments, the C3 Pathway is defined by the total abundance of genes in the metagenome that are identified by homology with the list of reference genes consisting of Idh, acrA, acrC, cat1, pduC, pduD, pduP, tesF, aarC, acrB, hsaG, Idh2, pudE.

In particular embodiments, the beneficial microbiome metagenomic function is the “C4 Pathway” associated with the production of butyrate and other short-chain fatty acids that provide direct nourishment for epithelial cells and promote a healthy inflammatory response by the animal. In particular embodiments, the C4 Pathway is defined by the total abundance of genes in the metagenome annotated by the E.C. numbers selected from the list of E.C. numbers consisting of 1.1.1.35, 1.1.1.36, 1.1.1.61, 1.2.1.76, 1.3.8.1, 2.3.1.247, 2.8.3.1; 2.8.3.8, 2.8.3.18, 2.8.3.9, 3.1.2.4, 4.2.1.150, 4.2.1.55, and 4.3.1.14. In particular embodiments, the C4 Pathway is defined by the total abundance of genes in the metagenome annotated by reactions selected from the list of reactions having the BioCyc reaction ID consisting of 2.8.3.9-RXN, 3-HYDROXBUTYRYL-COA-DEHYDRATASE-RXN, 3-HYDROXYISOBUTYRYL-COA-HYDROLASE-RXN, 4-HYDROXYBUTYRATE-DEHYDROGENASE-RXN. In particular embodiments, the C4 Pathway is defined by the total abundance of genes in the metagenome that are identified by homology with the list of reference genes consisting of 4hbD, atoA, atoD, cat1, crt1, ctfB, ech, fadB, fadE, fadJ, FOX2, pdaB, phaJ1, scr, yihU, aarC, abfH, bcd, cat3, crt, ctfA, kal, kce, paaZ, pdbB, and sucD.

IV. Targeted Delivery of Metabolites to the Gastrointestinal Tract A. Gastrointestinal Metabolites In certain embodiments, the methods described herein comprise delivering or increasing one or more gastrointestinal metabolites in a gastrointestinal tract of an animal. In some embodiments, one or more of the metabolites are detected and quantified. In some embodiments, the metabolites comprise short chain fatty acids (SCFAs), nitrogenous metabolites, metabolites of the carbon pathways, for example amino acids, pyruvic acid, butyric acid, propionic acid, acetic acid, lactic acid, valeric acid, isovaleric acid any combination thereof.

In some embodiments, one or more of the metabolites promote growth of the animal. In some embodiments, one or more of the metabolites promote growth of the animal and selected from the group consisting of: pyruvic acid, butyric acid, propionic acid, acetic acid, lactic acid, valeric acid, and isovaleric acid.

In a particular embodiment, the one or more metabolite comprise propanoate, pyruvic acid or both.

C. Metabolites Level

In some embodiments, the method of delivering or increasing one or more metabolites in a gastrointestinal tract of an animal comprises detecting the level of at least one of the one or more metabolites in the sample. In some embodiments, the method of delivering or increasing one or more metabolites in a gastrointestinal tract of an animal comprises detecting the level of at least 2, 3, 4, 5, 6, 7, 8, 9, or 10 metabolites in the sample. In some embodiments, the level of the metabolite is determined, in whole or in part, by LC or GC. In some embodiments, the level of the metabolite is determined, in whole or in part, by mass spectrometry. In some embodiments, the level of the metabolite is determined, in whole or in part, by NMR.

In certain embodiments, the level of the metabolites in a compartment in a gastrointestinal tract of the animal is detected. Accordingly, in certain embodiments, the level of the one or more metabolites in the same compartment is compared. In certain embodiments, the level of the one or more metabolites in different compartments is compared.

In some embodiments, a level of one or more metabolites in the gastrointestinal tract of the animal that is administered the nutritional composition comprising the SOD preparation is higher relative to a level of the metabolite in the gastrointestinal tract of an animal administered a nutritional composition lacking the SOD preparation.

For example, in some specific embodiments, the level of butyric acid in the gastrointestinal tract of the animal that is administered the nutritional composition comprising the SOD preparation is higher relative to a level of butyric acid in the gastrointestinal tract of an animal administered a nutritional composition lacking the SOD preparation. In some specific embodiments, the level of propionic acid in the gastrointestinal tract of the animal that is administered the nutritional composition comprising the SOD preparation is higher relative to a level of propionic acid in the gastrointestinal tract of an animal administered a nutritional composition lacking the SOD preparation. In some specific embodiments, the level of one or more essential oils in the gastrointestinal tract of the animal that is administered the nutritional composition comprising the SOD preparation is higher relative to a level of one or more essential oils in the gastrointestinal tract of an animal administered a nutritional composition lacking the SOD preparation.

In some embodiments, a level of 2, 3, 4, 5, 6, 7, 8, 9, 10, or more metabolites in the gastrointestinal tract of the animal that is administered the nutritional composition comprising the SOD preparation are each higher relative to a level of the metabolite in the gastrointestinal tract of an animal administered a nutritional composition lacking the SOD preparation.

In some embodiments, the detecting of the level of the one or more metabolites is performed after the administration of the nutritional composition. For example, in some embodiments, depending on the type and age of the animal, the level of the one or more metabolites is detected at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 2 days, or 3 days from the administration of the nutritional composition. In certain embodiments, the level of the one or more metabolites is detected at most 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 2 days, or 3 days from the administration of the nutritional composition.

V. Enhancing Animal Performance

A. Feed Conversion Ratio

In some embodiments, the methods described herein include reducing the feed conversion ratio of an animal. In some embodiments, an animal administered a SOD preparation, a nutritional composition, an animal feed pre-mix, or an animal feed composition as described herein has a lower feed conversion ratio compared to an animal provided a diet that does not include the SOD preparation.

B. Body Weight

In some embodiments, a subject animal that is fed a SOD preparation, nutritional composition, animal feed pre-mix, or animal feed composition described herein may experience an increase in weight gain, compared to a control animal that is not fed the SOD preparation, nutritional composition, animal feed pre-mix, or animal feed composition. In certain embodiments, both the subject animal and the control animal consume the same quantity of feed on a weight basis, but the subject animal provided the SOD preparation, nutritional composition, animal feed pre-mix, or animal feed composition experiences an increase in weight gain compared to the control animal that is fed a diet that does not include the SOD preparation.

VI. Administration

In some embodiments, administration comprises providing a SOD preparation, a nutritional composition, or an animal feed composition described herein, to an animal such that the animal may ingest the SOD preparation, the nutritional composition, or the animal feed composition at will. In such embodiments, the animal ingests some portion of the SOD preparation, the nutritional composition, or the animal feed composition.

The SOD preparation, nutritional composition, animal feed pre-mix, or animal feed composition may be provided to the animal on any appropriate schedule. In some embodiments, the animal is provided the SOD preparation, nutritional composition, animal feed pre-mix, or animal feed composition on a daily basis, on a weekly basis, on a monthly basis, on an every other day basis, for at least three days out of every week, or for at least seven days out of every month.

In some embodiments, the nutritional composition, the SOD preparation, the animal feed pre-mix, or the animal feed composition is administered to the animal multiple times in a day. For examples, in some embodiments, the nutritional composition, the SOD preparation, the animal feed pre-mix, or the animal feed composition is administered to the animal at least 1, 2, 3, 4, 5, 6, 7, 8, 9, or 10 times a day. In some embodiments, the nutritional composition, the SOD preparation, the animal feed pre-mix, or the animal feed composition is administered to the animal at most 1, 2, 3, 4, 5, 6, 7, 8, 9, or 10 times a day.

### EXAMPLES

### Example 1

Cloning and Expression of Superoxide Dismutase from Trichoderma reesei

### Strains

Escherichia coli Top-10 strain purchased from Invitrogen (Life Technologies, Carlsbad, CA, USA) was used to propagate our expression vectors encoding for lysozyme polypeptides. Aspergillus oryzae strain MT3568 was used for heterologous expression of the SOD polypeptide encoding sequences. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene with the pyrG gene.

Media

DAP2C-1 medium was composed of 0.5 g yeast extract, 30 g Maltodextrin, 11 g magnesium sulphate heptahydrate, 1 g dipotassium phosphate, 2 g citric acid monohydrate, 5.2 g potassium phosphate tribasic monohydrate, 1 mL Dowfax 63N10 (antifoaming agent), 2.5 g calcium carbonate, supplemented with 1 mL KU6 metal solution, and deionised water to 1000 mL.

KU6 metal solution was composed of 6.8 g ZnCl₂, 2.5 g CuSO₄·5H₂O, 0.13 g NiCl₂, 13.9 g FeSO₄·7H₂O, 8.45 g MnSO₄·H₂O, 3 g C₆H₈O₇·H₂O, and deionised water to 1000 mL.

YP 2% glucose medium was composed of 10 g yeast extract, 20 g Bacto-peptone, 20 g glucose, and deionised water to 1000 mL.

LB plates were composed of 10 g of Bacto-tryptone, 5 g of yeast extract, 10 g of sodium chloride, 15 g of Bacto-agar, and deionised water to 1000 mL.

LB medium was composed of 10 g of Bacto-tryptone, 5 g of yeast extract, and 10 g of sodium chloride, and deionised water to 1000 mL.

COVE-Sucrose-T plates were composed of 342 g of sucrose, 20 g of agar powder, 20 mL of COVE salt solution, and deionised water to 1000 mL. The medium was sterilized by autoclaving at 15 psi for 15 minutes (Bacteriological Analytical Manual, 8th Edition, Revision A, 1998). The medium was cooled to 60° C. and 10 mM acetamide, Triton X-100 (50 μL/500 mL) were added.

COVE-N-Agar tubes were composed of 218 g Sorbitol, 10 g Dextrose, 2.02 g KNO₃, 25 g agar, 50 mL Cove salt solution, and deionised water up to 1000 mL.

COVE salt solution was composed of 2 6 g of MgSO₄·7H₂O, 26 g of KCl, 26 g of KH₂PO₄, 50 mL of COVE trace metal solution, and deionised water to 1000 mL.

COVE trace metal solution was composed of 0.04 g of Na₂B₄O₇·10H₂O, 0.4 g of CuSO₄·5H₂O, 1.2 g of FeSO₄·7H₂O, 0.7 g of MnSO₄—H₂O, 0.8 g of Na₂MoO₄·2H₂O, 10 g of ZnSO₄·7H₂O, and deionised water to 1000 mL.

Cloning

Aspergillus niger MBin118 is disclosed in WO 2004/090155.

The SEQ ID NO 1 polypeptide coding sequence was cloned from Trichoderma reesei QM6a DNA by PCR.

Trichoderma reesei QM6a was cultivated in 100 ml of YP+2% glucose medium in 1000 ml Erlenmeyer shake flasks for 5 days at 20° C. Mycelia were harvested from the flasks by filtration of the medium through a Buchner vacuum funnel lined with MIRACLOTH® (EMD Millipore, Billerica, MA, USA). Mycelia were frozen in liquid nitrogen and stored at −80 C until further use. Genomic DNA was isolated using a DNEASY® Plant Maxi Kit (QIAGEN GMBH, Hilden Germany) according to the manufacturer's instructions.

Genomic sequence information was generated by Illumina MiSeq (Illumina Inc., San Diego, CA). 5 μs of the isolated Trichoderma reesei QM6a genomic DNA was used for library preparation and analysis according to the manufacturer's instructions. A 300 bp, paired end strategy was employed with a library insert size of 200-500 bp. The reads were subsequently fractionated to 25% followed by trimming (extracting longest sub-sequences having Phred-scores of 10 or more). These reads were assembled using Idba version 0.18. Contigs shorter than 200 bp were discarded. Genes were called using GeneMark.hmm ES version 2.3c and identification of the catalytic domain was made using “SOD_Cu” Hidden Markov Model provided by Pfam. A Swissprot entry of the identical sequence is also available: G0RPL7_HYPJQ. The polypeptide coding sequence for the entire coding region was cloned from Trichoderma reesei QM6a genomic DNA by PCR using the primers (SEQ ID NO: A and SEQ ID NO: B) described below.

5′-ACACAACTGGGGATCCACCATGCGGCCGTCTGGGTTCCT-3′ (SEQ ID NO: A)

5′-CTAGATCTCGAGAAGCTTTCACAGGGAGAAGAAGATGGC-3′ (SEQ ID NO: B)

Bold letters represent Trichoderma harzianum enzyme coding sequence. Restriction sites are underlined. The sequence to the left of the restriction sites is homologous to the insertion sites of pDau109 (WO 2005/042735).

In-Fusion™ Advantage PCR Cloning Kit Cat. nr 639620 The amplification reaction (50 μl) was performed according to the manufacturer's instructions (Thermo Scientific) with the following final concentrations:
1× Phusion HC buffer
200 uM dNTP
2.0 mM MgCl₂
0.5 uM of each primer of SEQ ID NO: A+B
10 ng of Trichoderma harzianum 04 genomic DNA.

The PCR reaction was incubated in a DYAD® Dual-Block Thermal Cycler (BioRad, USA) programmed for 1 cycle at 98° C. for 2 minutes; 30 cycles each at 98° C. for 10 seconds and 72° C. for two minutes followed by 1 cycle at 72° C. for 6 minutes. Samples were cooled to 10° C. before removal and further processing.

Five μl of the PCR reaction were analyzed by 1% agarose gel electrophoresis using 40 mM Tris base, 20 mM sodium acetate, 1 mM disodium EDTA (TAE) buffer. A major band of about 1 kb was observed. The remaining PCR reaction was purified directly with an ILLUSTRA™ GFX™ PCR DNA and Gel Band Purification Kit (GE Healthcare, Piscataway, NJ, USA) according to the manufacturer's instructions.

Two μg of plasmid pDau109 was digested with Bam HI and Hind III and the digested plasmid was run on a 1% agarose gel using 50 mM Tris base-50 mM boric acid-1 mM disodium EDTA (TBE) buffer in order to remove the stuffer fragment from the restricted plasmid. The bands were visualized by the addition of SYBR® Safe DNA gel stain (Life Technologies Corporation, Grand Island, NY, USA) and use of a 470 nm wavelength transilluminator. The band corresponding to the restricted plasmid was excised and purified using an ILLUSTRA™ GFX™ PCR DNA and Gel Band Purification Kit. The plasmid was eluted into 10 mM Tris pH 8.0 and its concentration adjusted to 20 ng per μl. An IN-FUSION® PCR Cloning Kit (Clontech Laboratories, Inc., Mountain View, CA, USA) was used to clone the 1450 bp PCR fragment into pDau109 digested with Barn HI and Hind III (20 ng). The IN-FUSION® total reaction volume was 10 μl. The IN-FUSION® total reaction volume was 10 μl. The IN-FUSION® reaction was transformed into FUSION-BLUE™ E. coli cells (Clontech Laboratories, Inc., Mountain View, CA, USA) according to the manufacturer's protocol and plated onto LB agar plates supplemented with 50 μg of ampicillin per ml. After incubation overnight at 37° C., transformant colonies were observed growing under selection on the LB plates supplemented with 50 μg of ampicillin per mL.

Several colonies were selected for analysis by colony PCR using the pDau222 pDau109 vector primers described below. Four colonies were transferred from the LB plates supplemented with 50 μg of ampicillin per ml with a yellow inoculation pin (Nunc A/S, Denmark) to new LB plates supplemented with 50 μg of ampicillin per ml and incubated overnight at 37° C.

Primer 8653:
(SEQ ID NO: C)
5′-GCAAGGGATGCCATGCTTGG-3′
Primer 8654:
(SEQ ID NO: D)
5′-CATATAACCAATTGCCCTC-3′

Each of the three colonies were transferred directly into 200 μl PCR tubes composed of 5 μl of 2× Thermo Scientific Dream Taq™ PCR Master Mix (Thermo Fisher Scientific, Rockford, IL, USA), 0.5 μl of primer 8653 (10 pm/μl), 0.5 μl of primer 8654 (10 pm/μl), and 4 of deionized water. Each colony PCR was incubated in a DYAD® Dual-Block Thermal Cycler programmed for 1 cycle at 94° C. for 60 seconds; 30 cycles each at 95° C. for 30 seconds, 60° C. for 45 seconds, 722C for 60 seconds, 682C for 10 minutes, and 10° C. for 10 minutes.

Four μl of each completed PCR reaction were submitted to 1% agarose gel electrophoresis using TAE buffer. All four E. coli transformants showed a PCR band of about 1 kb. Plasmid DNA was isolated from each of the four colonies using a QIAprep Spin Miniprep Kit (QIAGEN GMBH, Hilden Germany). The resulting plasmid DNA was sequenced with primers 8653 and 8654 using an Applied Biosystems Model 3730 Automated DNA Sequencer using version 3.1 BIG-DYE™ terminator chemistry (Applied Biosystems, Inc., Foster City, CA, USA).

One plasmid was chosen for transforming Aspergillus oryzae MT3568. A. oryzae MT3568 is an amdS (acetamidase) disrupted gene derivative of Aspergillus oryzae JaL355 (WO 2002/40694) in which pyrG auxotrophy was restored by inactivating the A. oryzae amdS gene. Protoplasts of A. oryzae MT3568 were prepared according to the method described in European Patent, EP0238023, pages 14-15.

E. coli 190 containing the selected plasmid was grown overnight according to the manufacturer's instructions (Genomed) and the plasmid DNA was isolated using a Plasmid Midi Kit (Genomed JETquick kit, cat.nr. 400250, GENOMED GmbH, Germany) according to the manufacturer's instructions. The purified plasmid DNA was transformed into Aspergillus oryzae MT3568. A. oryzae MT3568 protoplasts were prepared according to the method of Christensen et al., 1988, Bio/Technology 6: 1419-1422. The selection plates consisted of COVE sucrose with +10 mM acetamide+15 mM CsCl+TRITON® X-100 (50 μl/500 ml). The plates were incubated at 37° C. Briefly, 8 uls of plasmid DNA representing 3 ugs of DNA was added to 100 uls MT3568 protoplasts. 250 ul of 60% PEG solution was added and the tubes were gently mixed and incubate at 370 for 30 minutes. The mix was added to 10 ml of pre-melted Cove top agarose (The top agarose melted and then the temperature equilibrated to 40 C in a warm water bath before being added to the protoplast mixture). The combined mixture was then plated on two Cove-sucrose selection petri plates with 10 mM acetamide. The plates are incubated at 37° C. for 4 days. Single Aspergillus transformed colonies were identified by growth on the selection Acetimide as a carbon source. Each of the four A. oryzae transformants were inoculated into 750 μl of YP medium supplemented with 2% glucose and also 750 μl of 2% maltodextrin and also DAP4C in 96 well deep plates and incubated at 37° C. stationary for 4 days. At same time the four transformants were restreaked on COVE-2 sucrose agar medium.

Culture broth from the Aspergillus oryzae transformants were then analyzed for production of the SEQ ID NO 1 polypeptide by SDS-PAGE using NUPAGE® 10% Bis-Tris SDS gels (Invitrogen, Carlsbad, CA, USA) according to the manufacturer. Two bands at approximately 97 kDa and 45 kDa were observed for each of the Aspergillus oryzae transformants. One A. oryzae transformant producing the SEQ ID NO 1 polypeptide was selected and was cultivated in 1000 ml Erlenmeyer shake flasks containing 100 ml of DAP2C medium at 30° C. for 3 days with agitation at 150 rpm.

Using Aspergillus versicolor, Aspergillus deflectus, or Aspergillus egyptiacus, SEQ ID NO 2, 3, and 4 were similarly cloned and expressed.

### Example 2

### Growth, Cecal Microbiome, Blood, and Ileal Tissue Sampling of Commercial Swine Fed SOD Preparations

Swine were fed diets comprising SOD preparations of Example 1 to assess the effects of the presence of the SOD preparations on growth performance, health, and gut microbiome functional metagenomics versus birds fed a control diet not containing SOD preparations.

Diets:

Industry-standard corn-soy swine feeds, were manufactured according to industry practices and a three-phase feeding program with the diet constructions and nutrient specifications listed in Table 1.

TABLE 1
Compositions of the Control Diet

| | Starter Phase | Grower Phase |
|---|---|---|
| Component (wt %) | (days 0-21) | (days 21-42) |
| Corn | 61.992 | 67.769 |
| Soybean Meal (47.9% CP) | 27.030 | 22.118 |
| Animal & Vegetable Fat | 0.715 | 0.523 |
| DL-methionine | 0.071 | 0.022 |
| Limestone | 0.738 | 0.664 |
| Dicalcium phosphate | 2.802 | 2.301 |
| Mineral & Vitamin Premix | 0.075 | 0.075 |

Study Treatment Groups were assigned as described in Table 2.

TABLE 2
Study Treatment Groups

| | Dose SOD |
|---|---|
| Treatment Group | (SOD SEQ: ID 1 U/kg) |
| T1 (Control) | 0 |
| T2 | 1000 |
| T3 | 2000 |

Grow-Out:

Weaned CG32 male piglets were obtained and placed randomly into floor pens constructed in a swine house, with 5 piglets per pen and a stocking density of about 3 square foot per piglet. Pens were assigned randomly to treatment groups, with 12 statistical replicates per treatment and pen as the experimental unit.

For each pen, the bedding consisted of built-up litter top-dressed with fresh wood shavings. A standard commercial environmental and lighting program was employed. Starter and grower diets were fed as mash. All diets were provided ad libitum via automatic feeders in each pen Animals and housing facilities were inspected daily, including recording the general health status, feed consumption, water supply and temperature of the facility. Any mortalities were recorded daily. The total mass of consumed feed was recorded for each pen. Weight gain a were then determined for each pen according to standard practices.

Blood, Cecal Microbiome, and Heal Tissue Sampling:

On days 14 and 42, one piglet was selected randomly from each pen for blood, ileal tissue, and cecal sampling. The live weight of each sampled piglet was recorded. A blood sample was collected via wing puncture into vacutainer tubes and frozen following coagulation and serum separation. Ceca from each sampled piglet was then collected using standard veterinary methods. Cecal contents were transferred to 5 mL conical tubes, the weight of the cecal contents was recorded, and the contents were flash frozen to −80° C. A small ileal tissue sample was collected by resection from the intestinal wall, followed by prompt treatment with RNA-polymerase inhibitor.

### Example 3

### Gut Microbiome Sequencing and Functional Metagenomic Analysis of Broilers Fed SOD Preparations

The collective DNA of the cecal microbiome samples obtained from the piglet study of Example 2 was sequenced and analyzed to assess the effect of SOD preparations on the expression of microbiome metabolic pathways associated with central carbon and central nitrogen utilization.

The cecal sample tubes of Example 2 were thawed, DNA was extracted using standard methods and analyzed by whole genome shotgun sequencing on an Illumina NovaSeq instrument, with 1×100 bp reads. Raw sequencing reads were refined by: trimming (adapter, quality, length; CutAdapt). Taxonomic assignments were made against the MetaPhlAn2 (db_v20) database. For each cecal microbiome sample, the resulting sequence data was annotated for their metabolic functions and matched to the enzymes listed in Table 3.

TABLE 3

| EC | | | | Enz |
|---|---|---|---|---|
| Number | Substrate | Product | Control | #SOD |
| 1.1.1.28 | Pyruvic Acid | D-lactate | −0.23679 | 0.124213 |
| 2.8.3.— | D-Lactate | (R)-lactoyl-CoA | −5.2926 | −3.01151 |
| 4.2.1.54 | (R)-lactoyl-CoA | Acryloyl-CoA | −2.0349 | −1.19005 |
| 1.3.1.95 | Acryloyl-CoA | Propanoyl-CoA | N/A | N/A |
| 2.8.3.1 | Propanoyl-CoA | propanoate | −1.07205 | −0.58772 |

These are the genes, substrates, and products between the pyruvic acid midpoint, a common metabolite, and propanoate, a valuable molecule in animal production systems that can be produced by the hosts gut microbiome. The values recorded show a normalized values for enzymes derived from the microbiome within the animals serving as a control and normalized values for animals treated with two different enzymes. The more positive a number is, indicates that enzyme is present in higher quantities and can potentially contribute to greater flux with greater flux of the final molecule propanoate improving animal performance. Here it is show that Enz #(Superoxide Dismutase) improves flux across all enzymes over no treatment and had improved animal performance in body-weight gain.

In summary it can be concluded:
Propanoate is a valuable molecule linked to animal production performance metrics
Pyruvic acid to propanoate via acryloyl-CoA is a critical pathway for propanoate production in the microbiome
Feed supplied enzymes can modify/modulate the abundance of enzymes in the microbiome responsible for pathway to produce propanoate from pyruvic acid
Feed supplied enzymes can therefore improve performance of animal production via modulation of pathways present in the microbiome

### Example 4

### Core Microbiome Functions for Animal Health, Nutrition, and Welfare

Metagenomic pathways were identified with microbiome functions anticipated to have causative effects on the host animal's local and system biology based on known utility of the metabolites involved in the associated pathways.

The “C3 Pathway” microbiome pathway group was defined by the reactions in Table 4, whose associated metabolites generally promote host gluconeogenesis. Exemplary metabolites associated with the C3 microbiome pathway include (R)-lactate, (R)-lactoyl-CoA, (S)-lactate, (S)-propane-1,2,-diol, 1-propanal, acetate, acetyl-CoA, acryloyl-CoA, propanoate, propanoyl-CoA, and pyruvate.

The “Amine Biosynthesis” microbiome pathway group was defined by the reactions in Table 5, whose associated metabolites generally constitute amines that can be absorbed and utilized by the host animal. Exemplary metabolites associated with the Amine Biosynthesis microbiome pathway include (2S, 3S)-3-methylaspartate, (R)-3-(phenyl)lactate, (R)-3-(phenyl)lactoyl-CoA, (S)-3-aminobutanoyl-CoA, 2-oxoglutarate, 3-(4-hydroxyphenyl)pyruvate, 4-aminobutanal, 4-aminobutanoate, 4-guanidinobutanoate, 4-guanidinobutyraldehyde, 4-guanidobutryamide, 4-maleyl-acetoacetate, 5-aminopentanal, 5-aminopentanoate, 5-guanidino-2-oxopentanoate, Agmatine, Ammonia, Cadaverine, Cinnamate, cinnamoyl-CoA, coenzyme_A, formamide, homogentisate, L-beta-lysine, L-cystathionine, L-glutamate, L-glutamate-5-semialdehyde, L-histidine, L-homocysteine, L-lysine, L-methionine, L-ornithine, L-proline, L-serine, Mesaconate, N-carbamoylputrescine, N-formimino-L-glutamate, N2-succinyl-L-arginine, Pyruvate, succincate_semialdehyde, succinyl-CoA, and urea.

The “C4 Pathway” microbiome pathway group was defined by the reactions in Table 6, whose associated metabolites include butyric acid and related short-chain fatty acids. Exemplary metabolites associated with the C4 Pathway microbiome pathway include (3R)-3-hydroxybutanoyl-CoA, (R)-lactate, (R)-lactoyl-CoA, (S)-3-aminobutanoyl-CoA, (S)-3-hydroxy-isobutanoate, (S)-3-hydroxy-isobutanoyl-CoA, (S)-3-hydroxybutanoyl-CoA, (S)-5-amino-3-oxohexanoate, (S)-lactate, 4-hydroxybutanoate, Acetate, Acetoacetate, acetoacetyl-CoA, acetyl-CoA, butanoate, butanoyl-CoA, coenzyme_A, crotonyl-CoA, succinate, succincate_semialdehyde, and succinyl-CoA.

TABLE 4
Reactions defining the microbiome “C3 Pathway” group

| | Representative | |
|---|---|---|
| EC Number | Genes | Reaction BioCyc ID |
| 2.8.3.1 | aarC, cat1 | RXN-8807 |
| 2.8.3.1 | aarC, cat1 | RXN-8807 |
| 2.8.3.1 | aarC, cat1 | RXN-8807 |
| 2.8.3.1 | aarC, cat1 | RXN-8807 |
| 1.3.1.95 | acrB, acrA, acrC | RXN-8568 |
| 2.8.3.1 | aarC, cat1 | RXN-8807 |
| 2.8.3.1 | aarC, cat1 | RXN-8807 |

TABLE 5
Reactions defining the “Amine Biosynthesis”
microbiome pathway group

| | Representative | |
|---|---|---|
| EC_Number | Genes | Reaction_BioCyc_ID |
| 2.8.3.17 | fldA | 2.8.3.17-RXN |
| 2.8.3.17 | fldA | 2.8.3.17-RXN |
| 2.8.3.17 | fldA | 2.8.3.17-RXN |

TABLE 6
Reactions of the microbiome “C4 Pathway”

| | Representative | |
|---|---|---|
| EC_Number | Genes | Reaction_BioCyc_ID |
| 4.2.1.150 | crt, crt1, fadJ, ech, | RXN-11667 |
| | fadB | |
| 2.8.3.18 | aarC, cat1 | RXN-8807 |
| 2.8.3.1; 2.8.3.8 | cat3 | R11-RXN |
| 2.8.3.18 | aarC, cat1 | RXN-8807 |
| 2.8.3.18 | aarC, cat1 | RXN-8807 |

1. A method of improving nitrogen utilization in an animal, the method comprising:
a. administering a nutritional composition comprising a base nutritional composition and a SOD preparation to said animal,
b. wherein said SOD preparation comprises a polypeptide having superoxide dismutase (SOD) activity; and
c. wherein the level of a plurality of metabolites associated with enhanced nitrogen utilization is higher in a gastrointestinal sample from said animal compared to a gastrointestinal sample from a comparable control animal that has been administered a comparable nutritional composition comprising said base nutritional composition and lacking said SOD preparation.

2. The method of claim 1, wherein said plurality comprises at least 3, 4, 5, 6, 7, 8, 9, or 10 metabolites.

3. The method of claim 1, wherein said plurality of metabolites comprises at least 2, 3, 4, 5, 6, 7, 8, 9, or 10 metabolites selected from the group consisting of (2S, 3S)-3-methylaspartate, (R)-3-(phenyl)lactate, (R)-3-(phenyl)lactoyl-CoA, (S)-3-aminobutanoyl-CoA, 2-oxoglutarate, 3-(4-hydroxyphenyl)pyruvate, 4-aminobutanal, 4-aminobutanoate, 4-guanidinobutanoate, 4-guanidinobutyraldehyde, 4-guanidobutryamide, 4-maleyl-acetoacetate, 5-aminopentanal, 5-aminopentanoate, 5-guanidino-2-oxopentanoate, Agmatine, Ammonia, Cadaverine, Cinnamate, cinnamoyl-CoA, coenzyme_A, formamide, homogentisate, L-beta-lysine, L-cystathionine, L-glutamate, L-glutamate-5-semialdehyde, L-histidine, L-homocysteine, L-lysine, L-methionine, L-ornithine, L-proline, L-serine, Mesaconate, N-carbamoylputrescine, N-formimino-L-glutamate, N2-succinyl-L-arginine, Pyruvate, succincate_semialdehyde, succinyl-CoA, or urea.

4. The method of claim 1, wherein said plurality comprises at least 2, 3, 4, 5, 6, 7, 8, 9, or 10 metabolites selected from the group consisting of acetyl-CoA, ammonia, D-alanine, Formate, Fumarate, Glycine, L-2-amino-3-oxobutanoate, L-alanine, L-asparagine, L-aspartate, L-glutamate, L-isoleucine, N-formimino-L-glutamate, N-formyl-L-glutamate, N2-succinylglutamate, and Pyruvate.

5. The method of claim 1, wherein said plurality comprises at least 2, 3, 4, 5, 6, 7, 8, 9, or 10 metabolites selected from the group consisting of (3S,5S)-3,5-diaminohexanoate, (S)-3-methyl-2-oxopentanoate, (S)-5-amino-3-oxohexanoate, 2-oxoglutarate, acetyl-CoA, ammonia, D-alanine, Formate, Fumarate, Glycine, L-2-amino-3-oxobutanoate, L-alanine, L-asparagine, L-aspartate, L-glutamate, L-isoleucine, N-formimino-L-glutamate, N-formyl-L-glutamate, N2-succinylglutamate, and Pyruvate.

6. A method of improving carbon utilization in an animal, the method comprising:
a. administering a nutritional composition comprising a base nutritional composition and a SOD preparation to said animal,
b. wherein said SOD preparation comprises a polypeptide having superoxide dismutase (SOD) activity; and
c. wherein the level of a plurality of metabolites associated with improved carbon utilization in a gastrointestinal sample from said animal is higher compared to a comparable control animal that has been administered a comparable nutritional composition comprising said base nutritional composition and lacking said SOD preparation.

7. The method of claim 6, wherein said plurality comprises at least 3, 4, 5, 6, 7, 8, 9, or 10 metabolites.

8. The method claim 6, wherein said plurality comprises at least 2, 3, 4, 5, 6, 7, 8, 9, or 10 metabolites selected from the group consisting of (R)-lactate, (R)-lactoyl-CoA, (S)-lactate, (S)-propane-1,2,-diol, 1-propanal, acetate, acetyl-CoA, acryloyl-CoA, propanoate, propanoyl-CoA, and pyruvate.

9. The method of claim 6, wherein the level of said at least metabolite is at least about 0.5%, 1%, 2%, 3%, 4%, 5%, 10%, 15%, 20%, or 25% higher than the level in said gastrointestinal sample from a comparable control animal that has been administered a comparable nutritional composition comprising said base nutritional composition lacking said SOD preparation.

10. The method of claim 6, wherein the level of said at least metabolite is at least about 0.1 fold, 0.2 fold, 0.3 fold, 0.4 fold, 0.5 fold, 0.6 fold, 0.7 fold, 0.8 fold, 0.9 fold, 1 fold, 2 fold, 3 fold, 4 fold, 5 fold, 6 fold, 7 fold, 8 fold, 9 fold, or 10 fold higher than the level in said gastrointestinal sample from a comparable control animal that has been administered a comparable nutritional composition comprising said base nutritional composition lacking said SOD preparation.

11. The method of claim 1, wherein the polypeptide having superoxide dismutase (SOD) activity is an EC 1-15-1-1 SOD selected from the group consisting of a Cu-SOD, a Zn-SOD, a Mn-SOD, and an Fe-SOD.

12. The method of claim 11, wherein the polypeptide having superoxide dismutase activity is selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1.
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
e. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 52 to 170 of SEQ ID NO: 1.
f. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 55 to 136 of SEQ ID NO: 2;
g. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 12 to 149 of SEQ ID NO: 3;
h. a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 47 to 179 of SEQ ID NO: 4;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the sequence of SEQ ID NO: 5;
j. a polypeptide encoded by a polynucleotide that hybridizes under medium-high stringency conditions with coding sequence of SEQ ID NO: 5 or the full-length complement thereof;
k. a variant of the polypeptide of SEQ ID NO: 1, or SEQ ID NO: 2, or SEQ ID NO: 3, or SEQ ID NO: 4, or amino acid residues 52 to 170 of SEQ ID NO: 1, or amino acid residues 55 to 136 of SEQ ID NO: 2, or amino acid residues 12 to 149 of SEQ ID NO: 3, or amino acid residues 47 to 179 of SEQ ID NO: 4 comprising a substitution, deletion, and/or insertion at one or more (e.g., several) positions and that has superoxide dismutase activity; and
l. a fragment of the polypeptide of (a), (b), (c), (d), (e), (f), (g), (h), (i), (j), or (k) that has superoxide dismutase activity.


SEQUENCE LISTING (5 sequences)

SEQ ID NO: 1 - protein, 307 aa, Trichoderma reesei
  mat_peptide (52)..(170)
MRPSGFLAVLSTALVGYAAAKDAPVVIDNPQGVEFKASLPKKPFFAGAALVGNVKGSVSATAGPAGKGVRFRVQFENLPKTGGPFLYHIHEEPAVNGNCTSTLAHLDPYHRGETPACNASEPQTCQVGDLSGKYGKITSDPFVAEYFDLYTSLQPGSPAFFGNRSIVIHYANKTRLTCANFAKLPVPAATTVPSTTGAHHTAIHGPLATGGAQHNLTGSHNTTGHHNFTSPHRTTGAASPSDVYTLTSVVTVPGSPTTSSSLTTTDDAGSGGASATSSVVPFPGAASGNRVSLSLLSGVVAAIFFSL

SEQ ID NO: 2 - protein, 206 aa, Aspergillus versicolor
  mat_peptide (55)..(136)
MNLKFLPAAVLGLLISTTAAQDNNADTTDTDTNAPVITDNEPLSFHHASLLKKENTTVYGAITITTRRESAAVQVDVSIGGIPEGEYLNYHIHAARVPDDGNCYLTGAHLDPHGRGQEPPCTITAPQTCEVGDLSGKHGPAWAPAGEEFRATYSDFFLANTPGDDAYYGDLSWVVHGSNGDRLTCGNFDGFSGGGFGNWGGQKSKA

SEQ ID NO: 3 - protein, 154 aa, Aspergillus deflectus
  mat_peptide (12)..(149)
MAAVFAIANITGIVSGIVSFSQASPGTPVSVGGVLQGLAPGLHGFHVHESPISNGDCATAGSHFNPFHVNHGGPTAEVRHVGDLGNIQADDNGFSILNIQDTHIQLSGEQNILGLSVVIHADPDDLGLGGYPDSLTTGHAGARVACADIVVPGV

SEQ ID NO: 4 - protein, 188 aa, Aspergillus egyptiacus
  mat_peptide (47)..(179)
MYFKVFTGAFLGLVLAVSAQDSSTHAPVITNNMPISLYRATLLQKDNTTVYGGIFIGSQGSESASQIDIFLGGIPEGEHLNYHIHQYPVPENGNCYQTGAHLDPYGRGQTPPCDITAPHTCEVGDISGKHGPAFAPPGEVFSASFPDFFLSNNDDKPAFFGNRSFVVHGSGGERLTCGNFEEVDISND

SEQ ID NO: 5 - DNA, 1049 bp, Trichoderma reesei
  gene (1)..(1049)
atgcggccgtctgggttcctggcggtcctttccaccgccctcgttggctatgccgccgcgaaagacgctcctgttgtcattgacaatcctcaaggcgtcgagttcaaggccagtctgcccaagaagccctttttcgccggcgctgcgctggtgggcaatgtcaagggatcggtctctgctacggctggtcctgctggcaagggcgtccggttcagagtccagtttgaaaacttgcccaagacgggcggtcctttttgtgagtcctcttcttgctctcgactactttctgtgtttcatctcttgtctccggtctcacttcatctttccggtcaatggctatgtgtgtgcgccctcaaagtagctaaccaatgcccgtatgcagtgtaccacatccacgaggagccggccgtcaatggcaactgtacctcaacactggcccatctggacccgtaccatcgcggcgagacacctgcctgcaatgcttccgagccccagacgtgccaggtcggtgatctgagcggaaagtacggcaaaatcacgtcggatcccttcgtggccgagtactttgacctctacacgtctctccagcccggcagcccagccttcttcggcaaccgatcaatcgtgatccattacgcaaacaagacgcggctgacgtgcgccaactttgccaagctgccggtgcccgccgcaacgacggtccccagcaccaccggtgctcatcacactgccattcatgggcccctggctaccggtggcgctcaacataacctcaccggctctcacaacaccacaggccaccacaacttcacgagcccccatcgcaccacaggcgctgcctccccctcagacgtctacaccctcaccagcgtcgtcacggtccctggcagtcctaccacctcgtcgagcctcaccaccaccgacgacgctgggagtggtggtgccagcgccacgtcatctgttgttccctttccgggtgcagcatcgggaaacagagtgtccctttccctgctgagcggtgttgtagccgccatcttcttctccctgtg